(19) United States
(12) Patent Application Publication
(10) Pub. No.: US 2013/0101634 A1
(43) Pub. Date: Apr. 25, 2013

(54) COMPOSITIONS AND METHODS FOR REDUCING MICROBIAL OVERGROWTH IN THE SMALL INTESTINES

(71) Applicant: Deerland Enzymes, Inc., Kennesaw, GA (US)

(72) Inventors: John Deaton, Kennesaw, GA (US); Theodore Hersch, Atlanta, GA (US); Hilton Grant Dawson, North Fort Myers, FL (US)

(73) Assignee: DEERLAND ENZYMES, INC., Kennesaw, GA (US)

(21) Appl. No.: 13/650,633

(22) Filed: Oct. 12, 2012

Related U.S. Application Data
(60) Provisional application No. 61/546,089, filed on Oct. 12, 2011.

(51) Int. Cl. A61K 38/47; A61K 31/05; A61K 31/198; A61K 35/74

(52) U.S. Cl. CPC A61K 38/47; A61K 35/742; A61K 31/05; A61K 31/198
USPC 424/400; 424/93.1; 424/93.4; 424/93.46; 424/93.462

(57) ABSTRACT

An antimicrobial composition for reducing bacterial overgrowth in the small intestine is disclosed. The composition comprises a lytic enzyme combined with one or more anti-microbial essential oils, and a probiotic. Unlike broad spectrum antibiotics the disclosed composition does not enter the blood stream, does not destroy all intestinal microflora, and can be used on a continuing basis.

### CROSS-REFERENCE TO RELATED APPLICATIONS

This application claims benefit of U.S. Provisional Application No. 61/546,089, filed Oct. 12, 2011, which is hereby incorporated herein by reference in its entirety.

### FIELD

Disclosed herein are compositions that comprise one or more lytic enzymes and one or more antimicrobial essential oils and methods of using such compositions to reduce microbial overgrowth in the small intestines.

### BACKGROUND

The presence of small intestine bacterial overgrowth syndrome (SIBOS) has been determined in studies in over 30% of the patients diagnosed with irritable bowel syndrome and Crohn's disease. It is theorized that these conditions and SIBOS may be different expressions of a common pathogenic process. Other abnormalities of the gastrointestinal (GI) tract such as low gastric acid conditions, hypomotility of small intestine as in diabetes and scleroderma, and pancreatic insufficiency are also associated with, or contribute to, SIBOS. These conditions and SIBOS are all common in the elderly.

The small intestinal microflora of a healthy adult normally contains relatively small numbers of microorganisms. Total counts are generally 10⁴ or less per milliliter of fluid. SIBOS has been described as any condition in which the proximal part of the small intestine contains greater than 10⁵ microorganisms per milliliter for extended periods.

Symptoms of SIBOS include bloating, diarrhea, gastrointestinal pain, and constipation. Patients with SIBOS can have difficulty with absorption of proteins, fats, carbohydrates, and other micronutrients due to bacterial consumption of nutrients and production of toxic metabolites leading to direct injury to enterocytes in the small intestine.

The cause of the bacterial overgrowth in the small intestine is sometimes due to lack of acidity in the stomach, permitting bacteria from foods to inoculate the small intestines, or to an abnormality in the ileocecal valve, permitting bacteria from the large intestine to migrate into the small intestine.

Traditional treatment for SIBOS includes eradication of the bacterial overgrowth using antibiotics followed by dietary restriction from dairy products, gas producing vegetables, carbohydrates and fats to reduce the symptoms from the bacterial overgrowth. Digestive enzymes, antioxidants, and minerals are used to reduce intestinal inflammation and boost immunity. This eliminates the condition and resulting symptoms temporarily, but because the underlying cause is untreated, the condition often returns requiring continued frequent use of antibiotics. In addition, this method of treatment destroys the microflora in the lower intestine and can lead to development of antibiotic resistant bacteria, including an overgrowth of single toxic bacteria like Clostridia difficile, culminating in a severe type of Colitis.

Accordingly, there is a need for compositions and methods for treating SIBOS that are safe and effective and overcome the drawbacks of traditional treatments.

### SUMMARY

Disclosed are compositions having antimicrobial activity for the reduction of microbial overgrowth in the small intestine. The compositions comprise one or more lytic enzymes combined with one or more anti-microbial essential oils, and a probiotic to promote a microflora balance. The compositions can also include cofactors or chelating agents that break down the cell walls of bacteria, preventing growth and reducing the count of the targeted bacterial species.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing cell growth (O.D. 600 as a function of time) of E. coli in cell culture with control nutrient broth (X) or nutrient broth containing 1 mg/ml lyzozyme (diamond), 1 mg/ml citrate+1 mg/ml lyzozyme (circle), or 1 mg/ml lyzozyme+1 mg/ml EDTA (square).

FIG. 2 is a graph showing cell growth (O.D. 600 as a function of time) of Bacteroides fragilis in cell culture with control nutrient broth (circle) or nutrient broth containing 1 mg/ml lyzozyme (sqaure) or 1 mg/ml lyzozyme+1 mg/ml EDTA (diamond).

FIG. 3 is a graph showing cell growth (O.D. 600 as a function of time) of E. coli in cell culture with control nutrient broth (circle) or nutrient broth containing 1 mg/ml nisin (diamond) or 1 mg/ml nisin+1 mg/ml EDTA (square).

FIG. 4 is a graph showing cell growth (O.D. 600 as a function of time) of Bacteroides fragilis in cell culture with control nutrient broth (circle) or nutrient broth containing 1 mg/ml nisin (diamond) or 1 mg/ml nisin+1 mg/ml EDTA (square).

FIG. 5 is a graph showing cell growth (O.D. 600 as a function of time) of E. coli in cell culture with control nutrient broth (open circle) or nutrient broth containing 1 mg/ml cinnamon essential oil (square), 1 mg/ml oregano essential oil (X), 1 mg/ml peppermint leaf essential oil (diamond), 1 mg/ml clove essential oil (triangle), 1 mg/ml garlic essential oil (closed circle), or 1 mg/ml thyme essential oil (+).

FIG. 6 is a graph showing cell growth (O.D. 600 as a function of time) of B. fragilis in cell culture with control nutrient broth (open circle) or nutrient broth containing 1 mg/ml cinnamon essential oil (square), 1 mg/ml oregano essential oil (X), 1 mg/ml peppermint leaf essential oil (diamond), 1 mg/ml clove essential oil (triangle), 1 mg/ml garlic essential oil (closed circle), or 1 mg/ml thyme essential oil (+).

FIG. 7 is a graph showing cell growth (O.D. 600 as a function of time) of E. coli B (diamond, square) or E. coli C (X, circle) in cell culture with control nutrient broth (diamond, X) or nutrient broth containing 0.6 mg/ml antimicrobial composition (circle, square). The antimicrobial composition contained a combination of oregano, garlic, and thyme essential oils, EDTA, egg lysozyme, and Bacillus subtilis.

FIG. 8 is a graph showing cell growth (O.D. 600 as a function of time) of Bacteroides uniformis B (diamond, square) or Bacteroides fragilis (X, circle) in cell culture with control nutrient broth (diamond, X) or nutrient broth containing 0.6 mg/ml antimicrobial composition (circle, square). The antimicrobial composition contained a combination of oregano, garlic, and thyme essential oils, EDTA, egg lysozyme, and Bacillus subtilis.

FIG. 9 is an image of agar gel plates plated with 1×10³ E. coli after 6 hours of growth in nutrient broth containing 5% bovine serum albumin. The plate on the right also contained the antimicrobial composition.

FIG. 10 is an image of agar gel plates plated with 1×10⁶ B. fragilis after 6 hours of growth in nutrient broth containing 5% bovine serum albumin. The plate on the right also contained the antimicrobial composition.

FIG. 11 is a graph showing cell growth (O.D. 660 as a function of time) of Candida albicans in cell culture with control nutrient broth (X) or nutrient broth containing 0.6 mg/ml antimicrobial composition (square).

FIGS. 12A-12C are images of 3M Yeast and Mold plates plated with 1×10⁶ (FIGS. 12A and 12B) or 1×10³ (FIG. 12C)C. albicans after 4 hours of growth at 37° C. in nutrient broth containing 5% bovine serum albumin. The plates in FIGS. 12B and 12C also contained the antimicrobial composition.

### DETAILED DESCRIPTION

The materials, compositions, and methods described herein can be understood more readily by reference to the following detailed descriptions of specific aspects of the disclosed subject matter and the Examples and Figure included herein.

Before the present materials, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### DEFINITIONS

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “an enzyme” includes mixtures of two or more such enzymes, reference to “the probiotic” includes mixtures of two or more such probiotics, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. “About” can mean within 5% of the stated value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “5” is disclosed, then “about 5” is also disclosed.

Reference will now be made in detail to specific aspects of the disclosed materials, compounds, compositions, and methods, examples of which are illustrated in the accompanying Examples and Figures.

### Compositions

Disclosed herein are compositions having antimicrobial activity for the reduction of microbial overgrowth in the small intestine. The disclosed compositions have antimicrobial activity against organisms, such as bacteria and yeast, that are commonly associated with microbial overgrowth in the small intestine. Unlike broad spectrum antibiotics, the disclosed composition does not enter the blood stream and does not destroy all intestinal microflora. In addition, the disclosed composition can be a non-pharmaceutical, nutritional remedy that is safe to use repeatedly.

Lytic Enzymes

The composition comprises one or more lytic enzymes. Lytic enzymes that can be used in the disclosed composition include, but are not limited to, lysozyme, lysostaphin, zymolase, cellulase, mutanolysin, glycanases, proteases, mannase, and lactoperoxidase.

Lysozymes, also known as muramidase or N-acetylmuramide glycanhydrolase, are glycoside hydrolases, enzymes (EC 3.2.1.17) that damage bacterial cell walls by catalyzing hydrolysis of 1,4-beta-linkages between N-acetylmuramic acid and N-acetyl-D-glucosamine residues in a peptidoglycan and between N-acetyl-D-glucosamine residues in chitodextrins. Lysozyme is abundant in a number of secretions, such as tears, saliva, human milk, and mucus. It is also present in cytoplasmic granules of the polymorphonuclear neutrophils (PMN). Large amounts of lysozyme can be found in egg white. Therefore, in some embodiments, the lytic enzyme is egg lysozyme.

The amount and concentration of lytic enzyme used in the composition can be determined empirically based on the antimicrobial potential of the specific lytic enzyme(s) used in the composition. In some embodiments, the composition comprises about 10 mg to about 1000 mg of the one or more lytic enzyme per unit dose, including about 50 to about 500 mg, or about 75 to about 150 mg per unit dose. In some embodiments, the composition comprises about 30 wt % to about 40 wt % lytic enzyme per unit dose.

Antimicrobial Essential Oils

The composition can also comprise one or more antimicrobial essential oils. An “essential oil” refers to a concentrated liquid having the distinctive scent, or essence, of the plant from which it is extracted. Thus, the term “essential” is used herein only in the sense that the oil has the essence of the plant. Antimicrobial essential oils that can be used in the disclosed compositions include, but are not limited to, carvarcol, thymol, oils of ginger, cinnamon, mint, onion, black cumin, oregano, thyme, clove, garlic, eucalyptus, lavender, leleshwa, lemon, lemon myrtle, neem, cilantro, tea tree, and peppermint. In some embodiments, the one or more antimicrobial essential oils comprise oils of garlic, oregano, thyme, or any combination thereof. The antimicrobial essential oils can be used alone or in various combinations to achieve the desired microbial inhibition.

The amount and concentration of essential oil used in the composition can be determined empirically based on the antimicrobial potential of the specific essential oil, or combination of oils, used in the composition. In some embodiments, the composition comprises about 5 mg to about 500 mg of the one or more essential oils per unit dose, including about 5 to about 100 mg, or about 5 to about 10 mg per unit dose. In some embodiments, the composition comprises about 2 wt % to about 3 wt % of the one or more essential oils per unit dose.

Chelating Agents

The composition can also include cofactors or chelating agents (chelates) that break down the cell walls of bacteria, preventing growth and reducing count of the targeted bacterial species. The coupling of lysis with bacterial inhibition maintains reduced bacterial concentration. Chelating agents can be either synthetic or natural. ethylenediaminetetraacetic acid (EDTA), ethylene glycol tetraacetic acid (EGTA),diethylene triamine pentaacetic acid (DTPA, or pentetic acid), ethylenediamine-N,N′-bis(2-hydroxyphenylacetic acid) (EDDHA) and similar molecules are examples of synthetic chelating agents. Amino Acids are an example of natural chelates. Additional non-limiting examples of chelating agents that can be used in the disclosed compositions include citrates, phosphates, lactoferrin, gluconates (gluconic acid and salts thereof (sodium, potassium, calcium, zinc, ammonium, etc. . . . )), alpha-hydroxy fatty acids, palmitic acid, phytic acid, humic acid, gallic acid, bile extracts, bilirubin, and biliverdin.

The amount and concentration of chelating agent used in the composition can be determined empirically based on the antimicrobial potential of the specific chelating agent(s) used in the composition. In some embodiments, the composition comprises about 10 mg to about 100 mg of the one or more chelating agent per unit dose, including about 20 to about 75 mg, or about 30 to about 60 mg per unit dose. In some embodiments, the composition comprises about 10 wt % to about 20 wt % of the chelating agent per unit dose.

Probiotic

The composition optionally also contains a probiotic to promote microflora balance. The optional probiotic component of the disclosed compositions comprises live microorganisms that beneficially affect the host individual by displacement of undesirable or pathogenic bacteria and by creating an environment that resists colonization of the undesirable bacteria.

The probiotic of the disclosed composition can in some cases contain any microorganism, or combination of microorganisms, that are naturally present in gut microflora. While there are between 300 and 1000 different species of bacteria in the gut, 99% of the bacterial come from about 30 or 40 species. Most bacteria belong to the genera Bacteroides, Clostridium, Fusobacterium, Eubacterium, Ruminococcus, Peptococcus, Peptostreptococcus, and Bifidobacterium. Other genera, such as Escherichia and Lactobacillus, are present to a lesser extent.

The probiotic of the disclosed composition is preferably a sporogenic bacteria, such as a bacteria of the genera Bacillus. Spores being heat-stable allow the product to be stored at room temperature in a desiccated form without any deleterious effect on viability. A second advantage is that the spore is capable of surviving the low pH of the gastric barrier. Sporogenic bacteria can colonize the small intestine and then pass into the large intestine to return the intestinal tract to a healthy functional organ. Bacillus species have been used as probiotics for at least 50 years. Non-limiting examples of Bacillus species that can be used in the disclosed compositions include Bacillus subtilis, Bacillus clausii, Bacillus cereus, Bacillus coagulans and Bacillus licheniformis. In some cases, the sporogenic bacteria is Bacillus subtillis, Bacillus coagulans, or a combination thereof.

B. coagulans is a lactic acid-forming bacterial species. The organism exhibits characteristics typical of both genera Lactobacillus and Bacillus. B. coagulans is a Gram-positive rod, catalase positive, spore-forming, motile, a facultative anaerobe. However, B. coagulans may appear Gram-negative when entering the stationary phase of growth.

B. subtilis is a rod-shaped, Gram-positive, catalase-positive bacterium. B. subtilis has the ability to form a tough, protective endospore, allowing the organism to tolerate extreme environmental conditions. In some embodiments, the probiotic is the Bacillus strain B. subtilis 2335.

In some examples, the disclosed compositions comprise a probiotic organism that is a Lactobacillus species, such as L. acidophilus, L. amylovorus, L. brevis, L. casei, L. casei subsp. rhamnosus (Lactobacillus GG), L. caucasicus, L. crispatus, L. delbrueckii subsp. bulgaricus (L. bulgaricus), L. fermentum (L. fermenti), L. gasseri, L. helveticus, L. johnsonii, L. lactis, L. leichmannii, L. paracasei, L. plantarum, L. reuteri, and L. rhamnosus. In other examples, the disclosed compositions comprise a probiotic organism that is a Biifidobacterium species, such as B. adolescentis, B. bifidum, B. breve, B. infantis, B. lactis (B. animalis), B. licheniformis, and B. longum. In still other examples, the disclosed compositions comprise a probiotic organism that is a lactic acid bacteria such as Enterococcus faecium, Lactococcus lactis, Leuconstoc mesenteroides, Pediococcus acidilactici, Streptococcus thermophilus.

In some embodiments, the disclosed compositions can comprise a nonliving probiotic. For example, the disclosed compositions can comprise inulin, fructooligosaccharides, galactooligiosaccharides, xylooligosaccharides, polydextrose,lactulose, tagatose, isomaltooligosaccarides, soybean oligosaccharides, lactoferrin, proanthocyanins, or any combination thereof.

The amount and concentration of probiotic used in the composition can be determined empirically based on the potential of the specific probiotic to promote microflora balance. For example, the composition can contain about 1 billion to about 100 billion colony-forming units (CFUs) of probiotic per unit dose, including about 1 billion to about 10 billion or about 5 billion to about 7 billion CFUs of probiotic per unit dose. In some embodiments, the composition comprises about 2 wt % to about 3 wt % of the probiotic per unit dose.

Specific Combinations

The disclosed composition can comprise various combinations of the components discussed above. For example, the disclosed antimicrobial composition can comprise oils of garlic, oregano, and thyme; egg lysozyme; EDTA; and Bacillus subtillis. In some embodiments, a single 325 mg unit dosage of the disclosed composition comprises the following ingredients:

| | | | mg/capsule | % |
|---|---|---|---|---|
| Chelating agent | EDTA | | 50.00 | 15.38% |
| Lytic Enzyme | LysoZyme | | 110.00 | 33.85% |
| Essential Oil | phenol blend of: | | 35.0 | 10.77% |
| Granules | | | | |
| granulating agent | maltodextrin | 50.6% | 17.72 | 5.45% |
| binding agent | starch | 25.3% | 8.86 | 2.73% |
| | cawacrol (oregano oil) | 18.5% | 6.47 | 1.99% |
| essential oils | thymol (thyme oil) | 3.0% | 1.06 | 0.33% |
| | Eugenol (clove oil) | 2.5% | 0.89 | 0.27% |
| Priobiotic | Bacillus subtilus | | 7.5 | 2.31% |
| | (6 billion CFUs) | | | |
| Lubricant | Silica | | 3.0 | 0.92% |
| Filler | Microcrystalline | | 119.5 | 36.77% |
| | cellulose | | | |
| | | | 325.0 | 100% |

This composition can be formulated for oral administration. For example, the composition can comprise an enteric coating. The a single unit dose of the disclosed composition may be administered 1, 2, 3, 4, 5, or 6 times daily for an extended period of time. For example, a single unit dose of the disclosed composition may be administered three times daily for an indefinite period.

### Methods

Also disclosed is a methods of treating or preventing small intestine bacterial overgrowth syndrome (SIBOS) in a subject that is both safe and effective. The method comprises administering to a subject in need thereof an effective amount of a pharmaceutical or nutritional composition disclosed herein. In some cases, an effective amount is an amount sufficient to reduce the concentration of microorganisms in the small intestine to less than 10⁵ cells per milliliter, including between 10² cells per milliliter and 10⁴ cells per milliliter. In other cases, an effective amount is an amount necessary to reduce one or more symptoms of SIBOS.

The disclosed method can control an imbalance in the intestinal microflora by reducing the bacterial concentration and optionally supplementing it with beneficial bacteria (e.g., in spores) that can colonize the intestine and prevent pathogenic bacteria and colonic bacteria from growing in the intestines. The composition can be used regularly as a prophylactic to maintain a balance in the intestinal microflora without the adverse side effects of antibiotics.

The disclosed composition can significantly reduce the concentration of E. coli, Bacteroides, Candida and other microorganisms by lysis of the cell wall of the organism and by growth inhibition from the chelating agents and from the antimicrobial essential oils. The active ingredients of the disclosed method may be delivered to the small intestine using a protective enteric coating, which protects the ingredients from destruction by the acidity and protease in the stomach.

Essential oils alone can inhibit growth of certain bacteria. However, they are generally considered bacteriostatic rather than bactericidal. Used alone, essential oils can only maintain the bacterial concentration. By coupling the use of a lytic enzyme and optional chelating agent with a mixture of antimicrobial essential oils, the bacterial concentration is rapidly reduced by several orders of magnitude and is maintained at the lower level for a length of time. The optional sporogenic bacteria can repopulate the small and large intestines to maintain proper microbial balance.

Unlike broad spectrum antibiotics, the disclosed composition does not enter the blood stream and does not destroy all intestinal microflora and can be used on a continuing basis.

### Nutritional & Pharmaceutical Compositions, Administration, Dosage

The compositions described herein can be provided in a pharmaceutical or nutritional composition. Therefore, the disclosed composition can further comprise a nutritional or pharmaceutically acceptable carrier. Depending on the intended mode of administration, the pharmaceutical or nutritional composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include an effective amount of the compositions described herein optionally in combination with a biologically acceptable carrier and, in addition, can include other medicinal agents, vitamins, minerals, trace elements, antioxidants, flavorings, thickeners, chelating agents, binders, carriers, or diluents. By “biologically acceptable” is meant a material that is not biologically or otherwise undesirable, which can be administered to an individual along with the selected compositions without causing unacceptable biological effects or interacting in a deleterious manner with the other components of the nutritional composition in which it is contained.

As used herein, the term carrier encompasses any excipient, diluent, filler, salt, buffer, stabilizer, solubilizer, lipid, stabilizer, or other material well known in the art for use in nutritional or pharmaceutical formulations. The choice of a carrier for use in a composition will depend upon the intended route of administration for the composition. The preparation of nutritional acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of biologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). Further examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and animal oils (fish oil). Proper fluidity can be maintained, for example, by the use of a coating such as lecithin, by the maintenance of the required particle size in the case of dispersions and by the use of surfactants.

These compositions can also contain adjuvants such as preserving, wetting, emulsifying, and dispensing agents. Isotonic agents, for example, sugars, sodium chloride, and the like can also be included.

In preferred embodiments, the composition is administered orally by daily ingestion. Therefore, the disclosed composition can be formulated for oral administration. Solid dosage forms for oral administration of the compositions described herein include capsules, tablets, pills, powders, and granules. In such solid dosage forms, the compositions described herein is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents. Solid compositions of a similar type can also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art.

They can contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compositions described herein include nutritional or pharmaceutically acceptable emulsions, solutions, suspensions, syrups, and elixirs. In addition to the active compounds, the liquid dosage forms can contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Administration of the compositions described herein to an individual can be carried out using effective amounts of the compositions described herein for periods of time effective to reduce one or more symptoms of SIBOS. An individual can include both mammals and non-mammals. Mammals include, for example, humans; nonhuman primates, e.g. apes and monkeys; domesticated animals, such as felines or canines; cattle; horses; sheep; rats; mice; pigs; and goats. Non-mammals include, for example, fish and birds. In preferred embodiments, the individual is a human or a domesticated animal.

The effective amount of the compositions described herein can be determined empirically by one of ordinary skill in the art and includes exemplary dosage amounts for a mammal of from about 0.5 to about 200 mg/kg of body weight of active compound per day, which can be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 2 0 mg/kg of body weight of active compound per day, about 10 mg/kg of body weight of active compound per day, or about 5 mg/kg of body weight of active compound per day. The expression effective amount, when used to describe an amount of compound in a method, refers to the amount of a compound that achieves the desired pharmacological effect or other effect, for example an amount that results in bacterial inhibition or growth.

In some embodiments, the composition is administered in an effective amount to inhibit the growth of pathogenic and/or colonic bacteria within the small intestines. For example, the composition can be administered in an amount effective to reduce the concentration of E. coli, Bacteroides, or Candida, within the small intestine of the subject by at least 10%, at least 30%, or at least 50%.

In some embodiments, the composition is administered in an effective amount to reduce one or more symptoms of SIBOS. SIBOS is generally diagnosed by microbial investigation of jejunal aspirates. Non-invasive hydrogen and methane breath tests may also be used to diagnose SIBOS using glucose or lactulose. Therefore, in some examples, hydrogen glucose or lactulose breath tests are used to evaluate SIBOS symptoms.

Those of skill in the art will understand that the specific dose level and frequency of dosage for any particular subject can be varied and will depend upon a variety of factors, including the activity of the specific composition employed, the metabolic stability and length of action of that composition, the species, age, body weight, general health, sex and diet of the subject, the mode and time of administration, rate of excretion, active combination, and severity of the particular condition.

The examples below are intended to further illustrate certain aspects of the methods and compounds described herein, and are not intended to limit the scope of the claims.

### EXAMPLES

### Example 1

### Effect of the Antimicrobial Composition on Bacteria

Materials and Methods

Growth Curves Since bacteria grow exponentially, it is useful to plot the logarithm of the relative population size [y=ln(NIN0)] against time. Frozen permanents of each strain were streaked for single colonies using nutrient agar plates warmed prior to use. Single bacterial cells were restreaked on a nutrient agar plate at 37° C. for 24 h to 72 h depending on strain. Cells of several well-grown colonies on the plate were added to 50 ml of nutrient broth with shaking at 37° C. until the culture reached the desired optical density. Ingredients of interest were slowly added while stirring at 37° C. Timer was started at the beginning of addition; addition of material did not last longer than 30 seconds. Samples were taken at various time points and the optical density of the culture was obtained at 600 nm. All experiments were replicated twice.

Survival Plating:

Samples were taken six hours after addition of ingredient from experiments above and plated on selective media and incubated at optimal growth conditions for 24 to 72 hours depending on strain.

Enzymes:

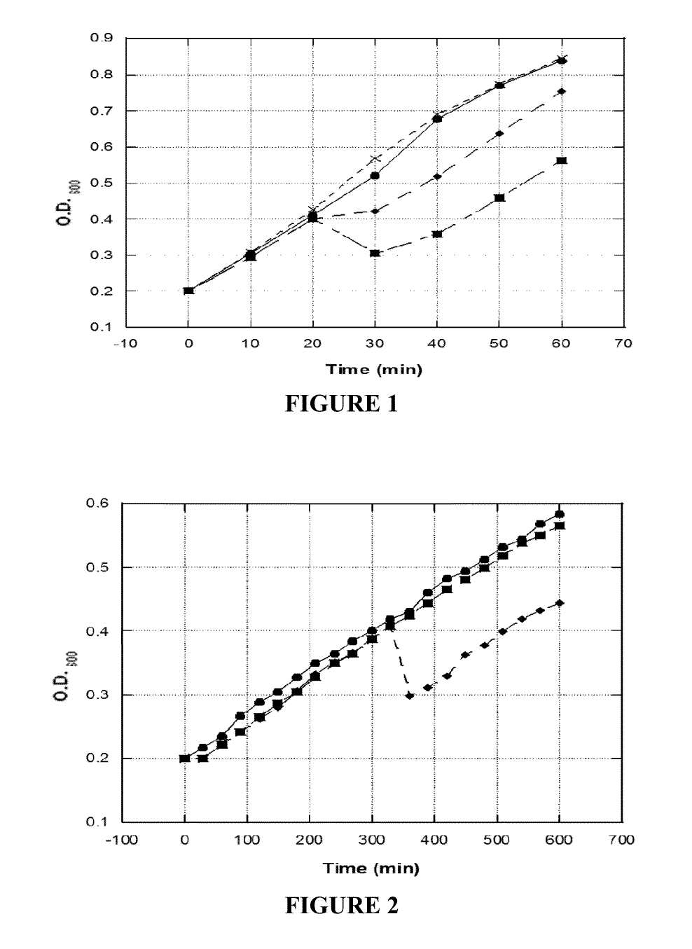

Lysozyme is an enzyme found in egg white, tears, and other secretions. It is responsible for breaking down the polysaccharide walls of many kinds of bacteria and yeast. Lysozyme is known to lyse gram positive bacteria but has difficulty accessing the cell wall of gram negative bacteria because of the extra protection given to the cell wall of gram negative bacteria by its outer membrane. However, the outer membrane is lined with sugars and requires metals to stabilize it. Therefore, a chelator such as EDTA or citrate can be combined with lysozyme to effectively kill E. coli, Bacteroides and Candida. However, cell growth is not totally eliminated and cell culture will recover (FIGS. 1 and 2).

Peptides:

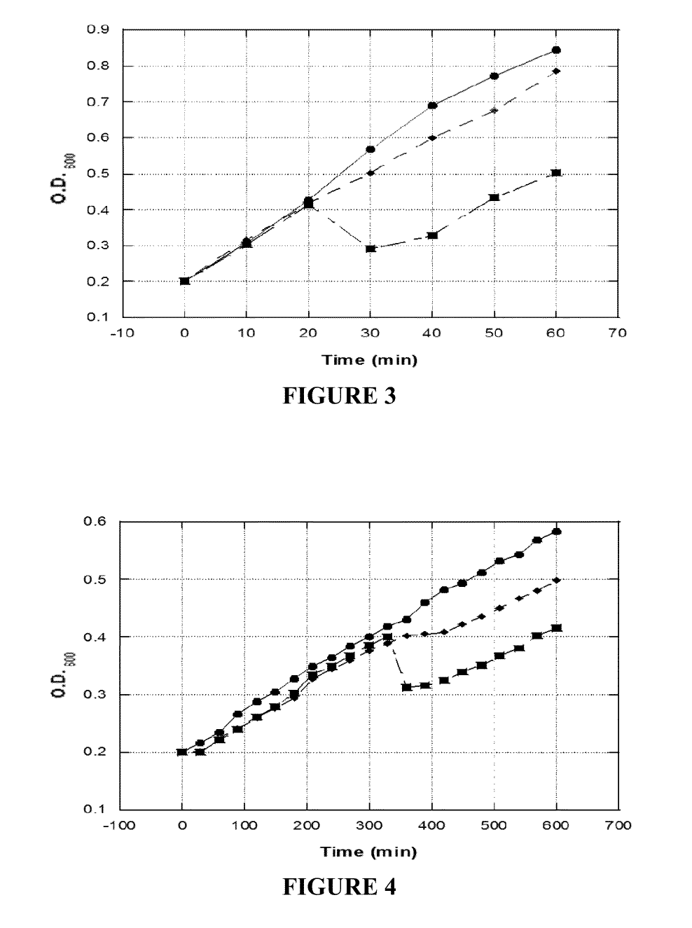

Nisin is an antibacterial peptide produced by Lactococcus lactis subsp. lactis that exhibits a broad spectrum of inhibitory activity against many colonic bacterial species. Escherichia coli exhibited nisin sensitivity when the outer membrane could be altered by treatments such as osmotic shock. In the United States, nisin has received GRAS (generally recognized as safe) status and is approved for use in some processed cheese spreads. In addition, nisin has been used to inactivate thermophilic spoilage organisms in canned goods and to extend the shelf life of milk and dairy products. The bactericidal action of nisin occurs in the cytoplasmic membrane of growing/multiplying cells. Bactericidal action can range from the loss of the proton motive force to the disruption of the cellular integrity of the membrane. As was observed with lysozyme, cell growth is not totally eliminated and cell culture will recover (FIGS. 3 and 4).

Chelators:

The outer membrane of gram-negative bacteria acts as a permeability barrier for the cell. It is responsible for preventing molecules such as antimicrobials, detergents, and dyes from reaching the cytoplasmic membrane. Gram-negative bacteria are not generally sensitive to wall degrading enzymes such as lysozyme or membrane destabilizing molecules such as nisin. Although the cytoplasmic membrane should be susceptible, the outer membrane protects the cell by excluding these particles. Magnesium ions serve to stabilize the lipopolysaccharide layer of the outer membrane. Chelating agents, such as EDTA, bind magnesium ions in the lipopolysaccharide layer and produce cells with increased susceptibility to antimicrobial molecules and detergents (FIGS. 1-4).

Essential Oils (EO):

The composition, structure as well as functional groups of the oils play an important role in determining their antimicrobial activity. Usually compounds with phenolic groups are most effective. Among these, the oils of clove, oregano, rosemary, thyme, sage and vanillin have been found to be most consistently effective against microorganisms. Most studies investigating the action of whole EOs against food spoilage organisms and food borne pathogens agree that, generally, EOs are generally more inhibitory against Gram positive than against Gram-negative bacteria. That gram-negative organisms are less susceptible to the action of antibacterials is perhaps to be expected, since they possess an outer membrane surrounding the cell wall, which restricts diffusion of hydrophobic compounds through its lipopolysaccharide covering. While this is true of many EOs, there are some such as oregano, clove, cinnamon and citral; which are effective against both groups. However, not all studies on EOs have concluded that gram-positives are more susceptible. There are also some non-phenolic constituents of oils such as allyl isothiocyanate, AIT, which are more effective or quite effective against Gram-negative bacteria as in garlic oil.

Results

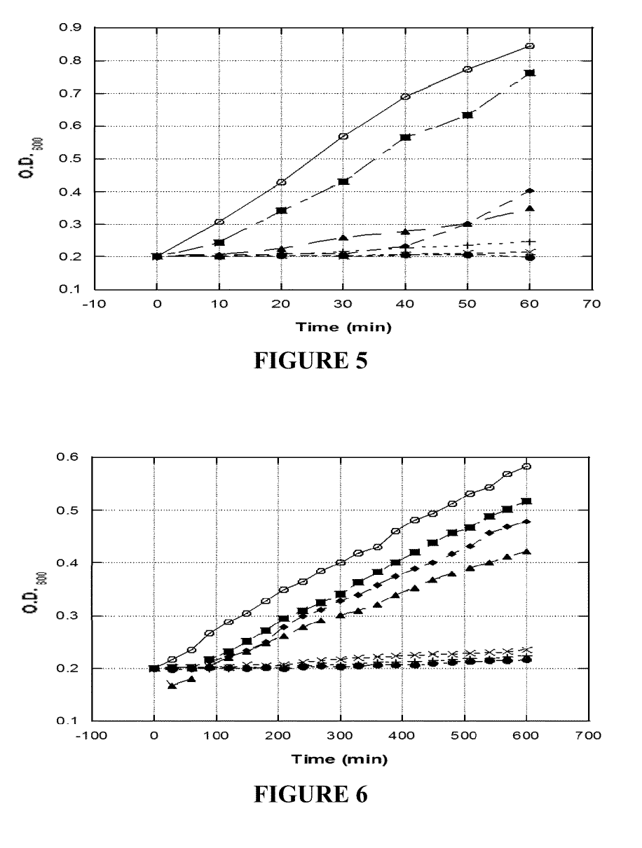

Numerous EOs were tested and found effective in inhibiting growth of bacteria in varying degrees. They did not reduce the colony density but reduced the rate of growth (FIGS. 5 and 6).

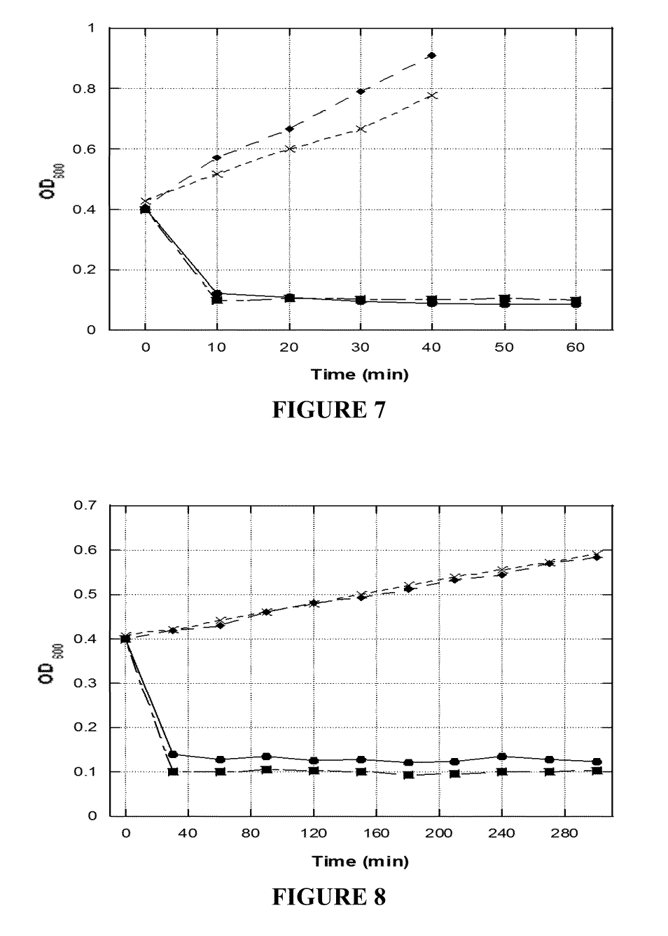

A composition containing a combination of EO (oregano, garlic and thyme), a chelating agent (EDTA), a Lytic enzyme (egg lysozyme), and a probiotic (bacillus subtilis) was evaluated utilizing the growth curve method. The composition was effective in reducing the cell density and was effective in inhibiting further growth. (FIGS. 7 & 8)

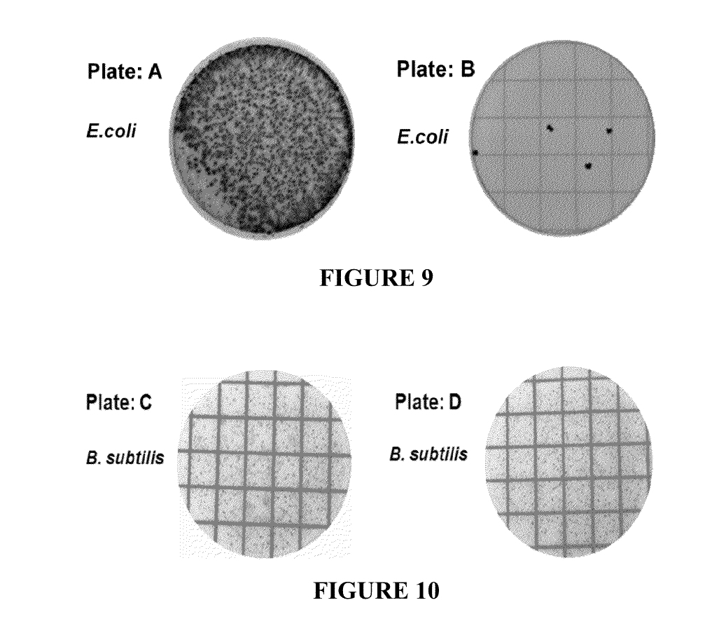

The composition was also tested by growing E. coli in nutrient broth containing 5% bovine serum albumin (used to simulate standard dirt load) for 6 hours with the Coctail added and without. The samples were diluted and plated on agar gel plates. Results indicated a marked reduction and suppression of the growth of E. coli by the antimicrobial composition (FIG. 9).

The compatibility of the probiotic Bacillus subtilis was examined by growing in nutrient broth containing 5% bovine serum albumin (used to simulate standard dirt load) for 6 hours with the composition added and without. The samples were diluted and plated on standard plates. These results confirm that B. subtilis growth is not affected in contrast with the marked reduction in suppression of the growth of E. coli (FIG. 10).

### Example 2

### Effect of the Disclosed Antimicrobial Composition on Yeast

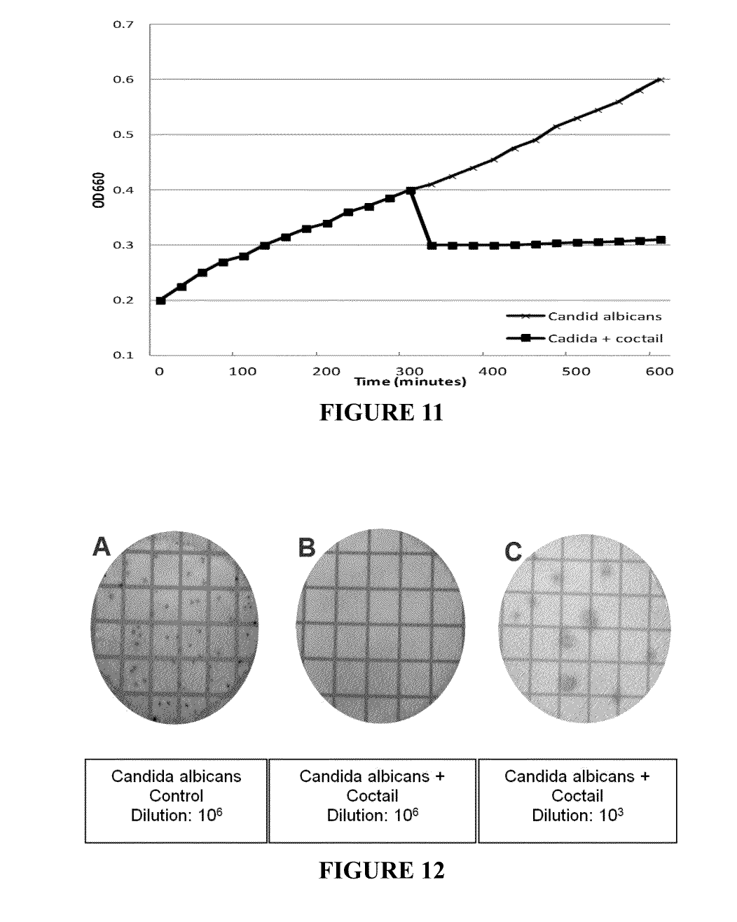

Candida Albicans was streaked on PDA (Potato dextrose agar) or on nutrient agar for single colonies. Single colony inoculants were started in 50 ml nutrient broth at pH 7.0 and 37° C. The Coctail of interest was added to one sample at 325 minutes. The OD 660 nm at time intervals for each sample was plotted showing significant reduction and inhibition of C. Albicans growth (FIG. 11).

Candida Albicans was streaked on PDA (Potato dextrose agar) or nutrient agar for single colonies. Single colony inoculants were started in 50 ml nutrient broth. Ingredients of interest were added at indicated time points and samples were allowed to grow for 4 more hours. Samples were diluted in phosphate buffer and plated at 1 thousand, 1 million and 1 billion on 3M Yeast and Mold plates and incubated at 30° C. The antimicrobial composition eliminated 99% of Candida yeast (FIG. 12).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. An antimicrobial composition, comprising:
(a) one or more lytic enzymes;
(b) one or more antimicrobial essential oils; and
(c) a probiotic.

2. The antimicrobial composition of claim 1, wherein the one or more lytic enzymes are selected from the group consisting of lysozyme, lysostaphin, zymolase, cellulase, mutanolysin, glycanases, proteases, mannase, and lactoperoxidase.

3. The antimicrobial composition of claim 2, wherein the one or more lytic enzymes comprise egg lysozyme.

4. The antimicrobial composition of claim 1, comprising about 10 mg to about 1000 mg of the one or more lytic enzymes per unit dose.

5. The antimicrobial composition of claim 4, comprising about 75 mg to about 150 mg of the one or more lytic enzymes per unit dose.

6. The antimicrobial composition of claim 1, wherein the one or more antimicrobial essential oils are selected from the group consisting of carvarcol, thymol, oils of ginger, cinnamon, mint, onion, black cumin, oregano, thyme, clove, garlic, eucalyptus, lavender, leleshwa, lemon, lemon myrtle, neem, cilantro, tea tree and peppermint.

7. The antimicrobial composition of claim 6, wherein the one or more antimicrobial essential oils comprise oils of garlic, oregano, thyme, or any combination thereof.

8. The antimicrobial composition of claim 1, wherein the composition comprises about 5 mg to about 500 mg of the one or more antimicrobial essential oils per unit dose.

9. The antimicrobial composition of claim 1, comprising about 5 mg to about 10 mg of the one or more antimicrobial essential oils per unit dose.

10. The antimicrobial composition of claim 1, wherein the probiotic is a sporogenic bacteria.

11. The antimicrobial composition of claim 10, wherein the sporogenic bacteria is selected from the group consisting of Bacillus subtilis, Bacillus clausii, Bacillus cereus, Bacillus coagulans and Bacillus licheniformis.

12. The antimicrobial composition of claim 10, wherein the sporogenic bacteria comprises Bacillus subtillis, Bacillus coagulans, or a combination thereof.

13. The antimicrobial composition of claim 1, comprising about 1 billion to about 100 billion colony-forming units (CFUs) of probiotic per unit dose.

14. The antimicrobial composition of claim 13, comprising about 5 billion to about 7 billion CFUs of probiotic per unit dose.

15. The antimicrobial composition of claim 1, further comprising a chelating agent.

16. The antimicrobial composition of claim 15, wherein the chelating agent is selected from the group consisting of a citrate, a phosphate, ethylenediaminetetraacetic acid (EDTA), and lactoferrin.

17. The antimicrobial composition of claim 1, comprising about 10 mg to about 100 mg of the chelating agent per unit dose.

18. The antimicrobial composition of claim 17, comprising about 30 mg to about 60 mg of the chelating agent per unit dose.

19. The antimicrobial composition of claim 1, comprising
(a) egg lysozyme;
(b) one or more essential oils selected from the group consisting of oils of garlic, oregano, and thyme;
(c) EDTA, and
(d) Bacillus subtillis.

20. The antimicrobial composition of claim 1, further comprising a pharmaceutically acceptable carrier.

21. The antimicrobial composition of claim 20, formulated for oral administration.

22. The antimicrobial composition of claim 21, wherein the composition comprises an enteric coating.

23. A method of treating or preventing small intestine bacterial overgrowth syndrome (SIBOS) in a subject, comprising administering to the subject the composition of claim 1 in an amount effective to inhibit the growth of pathogenic and/or colonic bacteria within the small intestine.

24. The method of claim 23, wherein the subject is a human subject

25. The method of claim 23, wherein the subject is a domesticated animal

26. The method of claim 25, wherein the subject is a canine.

27. The method of claim 23, wherein the composition is administered prophylactically to the subject to maintain a balance in the intestinal microflora.

28. The method of claim 23, wherein the composition is administered orally by daily ingestion.

29. The method of claim 23, wherein the composition is administered in an amount effective to reduce the concentration of microorganisms in the proximal part of the small intestine to less than 10⁵ cells per milliliter.
